(19) United States
(12) Patent Application Publication
(10) Pub. No.: US 2015/0265644 A1
(43) Pub. Date: Sep. 24, 2015

(54) THERAPEUTIC METHODS AND COMPOSITIONS FOR TREATING DIABETES UTILIZING DITERPENOID COMPOUNDS

(71) Applicant: Orient Europharma Co., Ltd., Taipei (TW)

(72) Inventors: Ming-Jai Su, Taipei City (TW); Shoei-Sheng Lee, Taipei City (TW)

(73) Assignee: Orient Europharma Co., Ltd., Taipei (TW)

(21) Appl. No.: 14/221,225

(22) Filed: Mar. 20, 2014

(51) Int. Cl. A61K 31/7048

(52) U.S. Cl. CPC A61K 31/7048

(57) ABSTRACT

The present invention is directed to therapeutic methods and compositions for treating insulin resistant diabetes or diabetes with inflammation in a subject comprising administering an effective amount of borapetoside A, or a pharmaceutically acceptable salt, metabolite, solvate or prodrug thereof, to said subject.

### BACKGROUND OF THE INVENTION

Diabetes mellitus (DM), or simply diabetes, is a group of metabolic diseases in which a person has high blood sugar, either because the pancreas does not produce enough insulin, or because cells do not respond to the insulin that is produced. This high blood sugar produces the classical symptoms of polyuria (frequent urination), polydipsia (increased thirst), and polyphagia (increased hunger).

There are three main types of diabetes mellitus (DM). Type 1 DM results from the body's failure to produce insulin, and currently requires the person to inject insulin or wear an insulin pump. This form was previously referred to as “insulin-dependent diabetes mellitus” (IDDM) or “juvenile diabetes”. Type 2 DM results from insulin resistance, a condition in which cells fail to use insulin properly, sometimes combined with an absolute insulin deficiency. This form was previously referred to as non insulin-dependent diabetes mellitus (NIDDM) or “adult-onset diabetes”. The third main form, gestational diabetes, occurs when pregnant women without a previous diagnosis of diabetes develop a high blood glucose level. It may precede development of type 2 DM.

The classic symptoms of untreated diabetes are loss of weight, polyuria (frequent urination), polydipsia (increased thirst), and polyphagia (increased hunger). Symptoms may develop rapidly (weeks or months) in type 1 diabetes, while they usually develop much more slowly and may be subtle or absent in type 2 diabetes. Prolonged high blood glucose can cause glucose absorption in the lens of the eye, which leads to changes in its shape, resulting in vision changes. Blurred vision is a common complaint leading to a diabetes diagnosis. A number of skin rashes that can occur in diabetes are collectively known as diabetic dermadromes.

Diabetes mellitus is a chronic disease, for which there is no known cure except in very specific situations. Management concentrates on keeping blood sugar levels as close to normal (“euglycemia”) as possible, without causing hypoglycemia. This can usually be accomplished with diet, exercise, and use of appropriate medications (insulin in the case of type 1 diabetes; oral medications, as well as possibly insulin, in type 2 diabetes). Metformin is generally recommended as a first line treatment for type 2 diabetes, as there is good evidence that it decreases mortality. Type 1 diabetes is typically treated with combinations of regular and NPH insulin, or synthetic insulin analogs. When insulin is used in type 2 diabetes, a long-acting formulation is usually added initially, while continuing oral medications. Doses of insulin are then increased to effect.

### SUMMARY OF THE INVENTION

In one aspect provided herein are methods for treating insulin resistant diabetes in a subject comprising administering an effective amount of borapetoside A, or a pharmaceutically acceptable salt, metabolite, solvate or prodrug thereof, to said subject.

In another aspect provided herein are methods for treating diabetes in a subject wherein said subject is in a inflammatory condition comprising administering said subject an effective amount of borapetoside A, or a pharmaceutically acceptable salt, metabolite, solvate or prodrug thereof, to said subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows chemical structure of borapetoside A.

FIG. 2A-C show illustrative effective results of Borapetoside A enhancing glycogen synthesis in C2C12 and Hep3B cells. Borapetoside A treatment increased the glycogen content in C2C12 (2A), IL-6-treated-C2C12 (2B), and Hep3B (2C). All data were presented as mean±SEM with three independent experiments. (*, p<0.05, compared with vehicle control.)

FIG. 3A-C show illustrative hypoglycemic effects of borapetoside A in mice. The mice were injected with borapetoside A, vehicle only, or positive drug intraperitoneally. Sixty minutes after injection, blood samples were collected from the mice and subjected to plasma glucose assay. The experiment was independently repeated for seven times, and the data were presented as mean±SEM in the normal mice (3A), the mice with type 1 DM (3B), and the mice with type 2 DM (3C). Asterisks indicate significant difference compared with vehicle only (*, p<0.05).

FIG. 4A-D show effects of borapetoside A on plasma glucose concentration and the AUC in normal mice (4A/4B) and the mice with type 2 DM (4C/4D) that received an IPGIT. The blood samples were obtained before (at 0 min) and after glucose injection (at 30, 60, 120, and 150 minutes) in the normal mice and the mice with type 2 DM (4A and 4C). The AUC decreased in the borapetoside A-treated group, as compared to the vehicle-treated group in both normal and T2DM mice (4B and 4D). Asterisk symbols indicate significant difference between vehicle and borapetoside A-treated groups at the same time point. Data were expressed as mean±SEM in each group (n=6). *, p<0.05, vs animals treated with vehicle.

FIG. 5A-C show illustrative effects of borapetoside A on glycogen synthesis in skeletal muscle in vivo. The normal mice (SA), the mice with type 1 DM (5B), and the mice with type 2 DM (SC) were used in this study. Values expressed as mean±SEM from six animals in each group. The average value of glycogen content of the vehicle-treated mice in each group was seen as 100. *, p<0.05, compared animals treated with vehicle control.

FIG. 6A-C show illustrative effects of borapetoside A on glycogen synthesis in liver in vivo. The normal mice (6A), the mice with type 1 DM (6B), and the mice with type 2 DM (6C) were used in this study. Values expressed as mean±SEM from six animals in each group. The average value of glycogen content of the vehicle-treated mice in each group was seen as 100. *, p<0.0.05, compared animals treated with vehicle control.

FIG. 7A-F show various protein expression levels in the liver of the mice with type 1 DM after 7-day treatment. The protein expression of IR-related signaling mediators was analyzed by immunoblot in the liver of the mice with type 1 DM after repeated intraperitoneal administrations of borapetoside A or insulin for 7 days (7A). The mice that did not receive any treatment were given the same volume of vehicle. The findings were reproduced on 3 separate experiments. The quantification protein levels expressed are expressed as mean. SEM in each column, as shown in (7B) to (7F). (*, p<0.05 compared with vehicle-treated.)

### DETAILED DESCRIPTION OF THE INVENTION

In accordance with the practice of this invention, borapetoside A is provided herein as a pharmacological agent for treatment of diabetes.

Herbal prescriptions have been recognized as potential remedy based on the scientific medical assessments. The hypoglycemic action and other medicinal uses of T. crispa extract were reported in several previous studies. However, the active ingredients in the extract are not well investigated.

In accordance with the practice of this invention, single dose administration of borapetoside A decrease the serum glucose levels and increases the insulin secretion in the normal mice and Type 2 DM bearing mice. Furthermore, borapetoside A attenuated the elevation of plasma glucose induced by intraperitoneal glucose in the normal mice and the mice with diabetes. The continuous treatment with borapetoside A for 7 days induced the phosphorylation of IR, Akt, AS160, and GLUT2, and decreased the expression of phosphoenolpyruvate carboxykinase (PEPCK) in the liver of the Type 1 DM bearing mice.

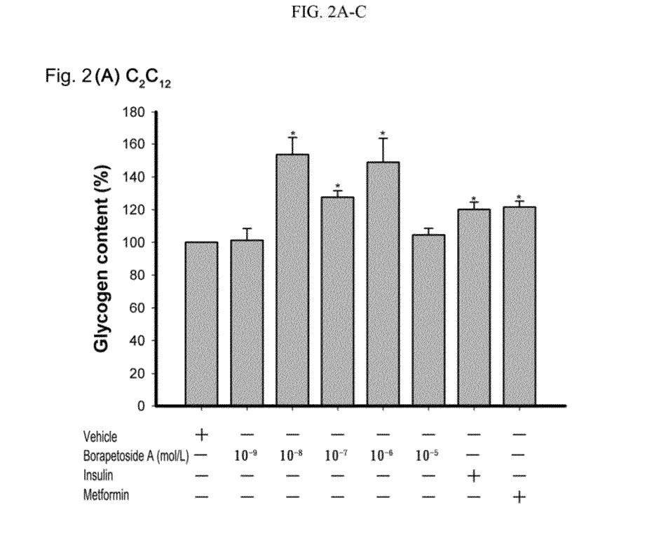

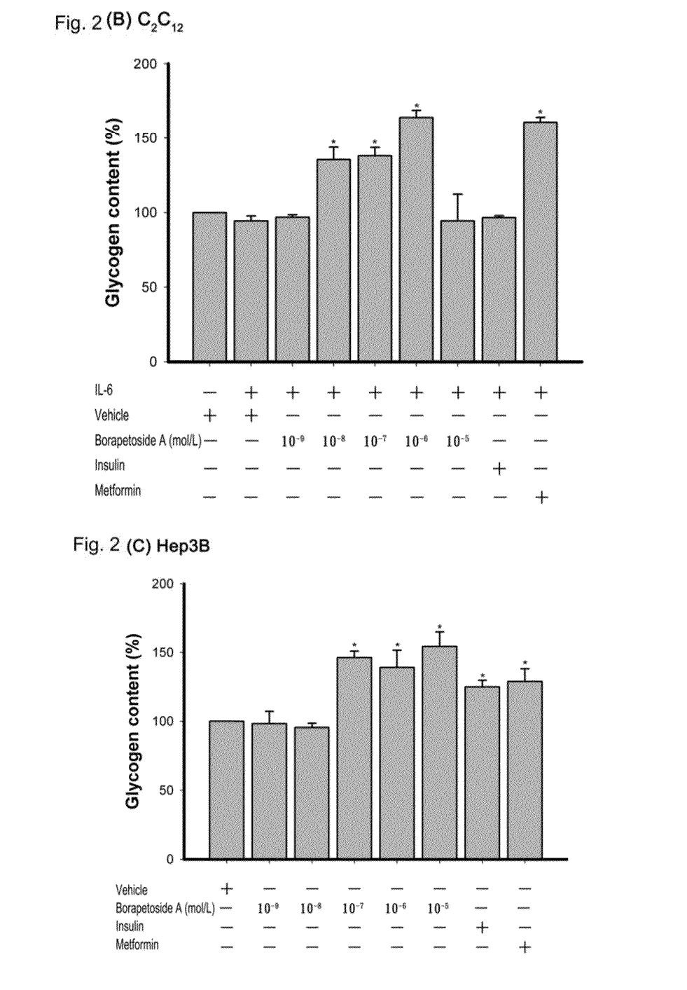

Specifically, it was found that borapetoside A effectively increased the glycogen content in C2C12 skeletal muscle cells and human hepatocellular carcinoma cell lines Hep3B at very low concentration ranging from 10⁻¹ to 10⁻⁷ mol/L. Since glycogen content in the IL-6-treated C2C12 cells was not increased with the insulin treatment, this result indicated that insulin resistance in C2C12 cells was induced after IL-6 treatment. In contrast, borapetoside A treatment enhanced the glycogen synthesis in IL-6-treated C2C12 cells (see e.g., FIG. 2). This result revealed that borapetoside A could still induce similar increase of glycogen content in these insulin-resistant C2C12 cells.

Interleukin 6 (IL-6) is an interleukin that acts as both a pro-inflammatory cytokine and an anti-inflammatory myokine. In humans, it is encoded by the 11.6 gene. IL-6 is an important mediator of fever and of the acute phase response. It is capable of crossing the blood-brain barrier and initiating synthesis of PGE₂ in the hypothalamus, thereby changing the body's temperature setpoint. In muscle and fatty tissue, IL-6 stimulates energy mobilization that leads to increased body temperature. IL-6 can be secreted by macrophages in response to specific microbial molecules, referred to aspathogen-associated molecular patterns (PAMPs). These PAMPs bind to an important group of detection molecules of the innate immune system, called pattern recognition receptors (PRRs), including Toll-like receptors (TLRs). These are present on the cell surface and intracellular compartments and induce intracellular signaling cascades that give rise to inflammatory cytokine production.

Interleukin (IL)-6 is one of several proinflammatory cytokines that have been associated with insulin resistance and type 2 diabetes. A two- to threefold elevation of circulating IL-6 has been observed in these conditions. It has been established that IL-6 can inhibit insulin receptor (IR) signal transduction and insulin action in both primary mouse hepatocytes and the human hepatocarcinoma cell line, HepG2. This inhibition depends on duration of IL-6 exposure, with a maximum effect at 1-1.5 h of pretreatment with IL-6 in both HepG2 cells and primary hepatocytes. The IL-6 effect is characterized by a decreased tyrosine phosphorylation of IR substrate (IRS)-1 and decreased association of the p85 subunit of phosphatidylinositol 3-kinase with IRS-1 in response to physiologic insulin levels. In addition, insulin-dependent activation of Akt, important in mediating insulin's downstream metabolic actions, is markedly inhibited by IL-6 treatment. Finally, a 1.5-h preincubation of primary hepatocytes with IL-6 inhibits insulin-induced glycogen synthesis by 75%. These data suggest that IL-6 plays a direct role in insulin resistance at the cellular level in both primary hepatocytes and HepG2 cell lines and may contribute to insulin resistance and type 2 diabetes.

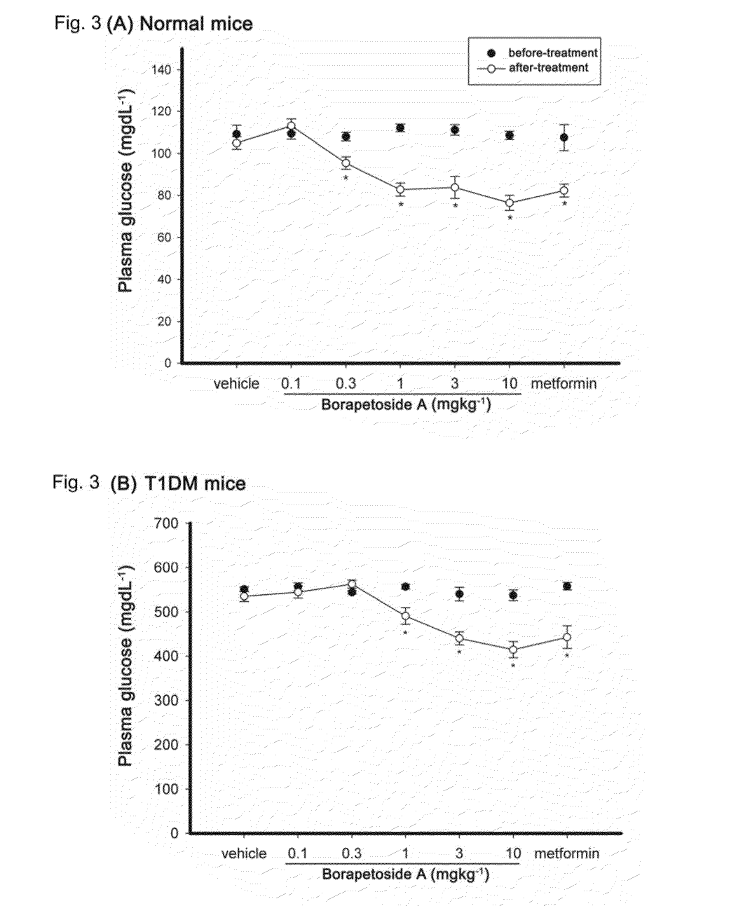

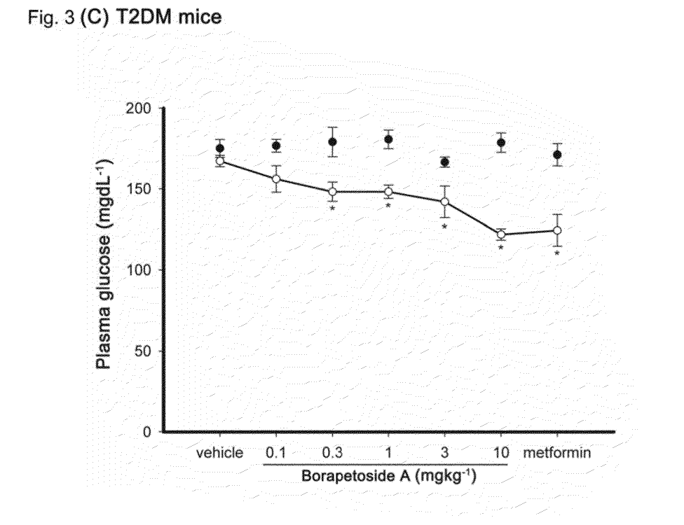

In accordance with the practice of this invention, it was found that bolus intraperitoneal injection of borapetoside A significantly lowers plasma glucose concentration in a dose-dependent manner in the normal mice, or the mice with diabetes (FIG. 3). The plasma glucose lowering effect of 10 mgkg⁻¹ borapetoside A was similar to the effect of 300 mgkg⁻¹ metformin. The increased plasma insulin level may contribute to the plasma glucose lowering effect of higher dose borapetoside A (3 and 10 mgkg⁻¹) in the normal mice; however, the plasma insulin in the normal mice was not significantly increased with 0.3 and 1 mgkg⁻¹ borapetoside A-treatment. Furthermore, in the mice with type 1 DM, borapetodie A did not alter the insulin level (Table 1) but significantly decreased the plasma glucose level (FIG. 3). In some embodiments there are provided that borapetoside A exert the hypoglycemic action through insulin-independent mechanism both in normal and mice with diabetes. The effective dose of borapetoside A (0.3 to 1 mgkg⁻¹) to exert insulin-independent hypoglycemic action is consistent with its effective concentration to stimulate glycogen synthesis in insulin-resistant C2C12 cells.

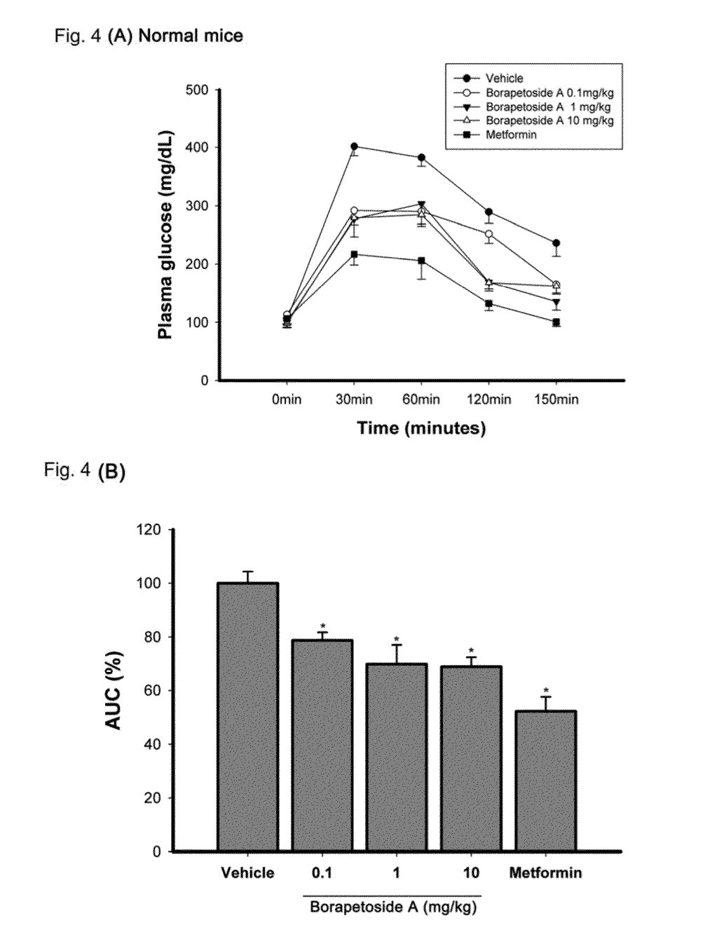

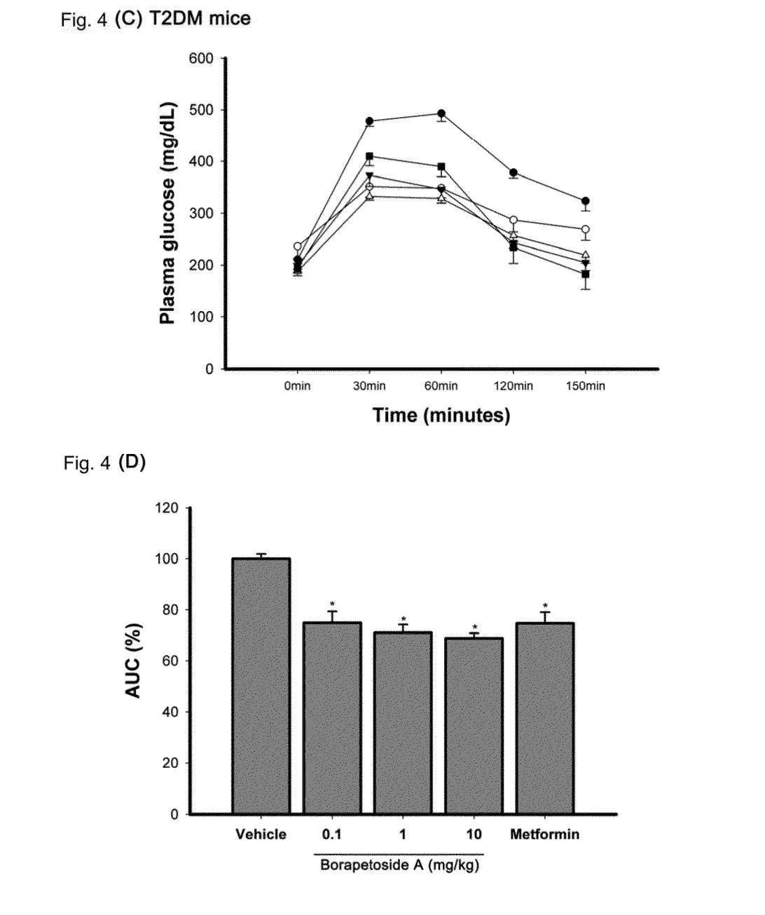

Glucose tolerance test is one of the most critical criteria for evaluating the therapeutic efficacy of hypoglycemic drugs. Described herein, the effect of borapetoside A on the glucose utilization was further verified by IPGrTT test. Borapetoside A has notably enhanced the glucose uptake and utilization in peripheral tissues, which is a major site of glucose disposal (Hollenbeck and Reaven, 1987), in the normal mice and the mice with type 2 DM mice (FIG. 4). Even in the ineffective dose of bolus test, 0.1 mgkg⁻¹ borapetoside A significantly decreased the AUC.

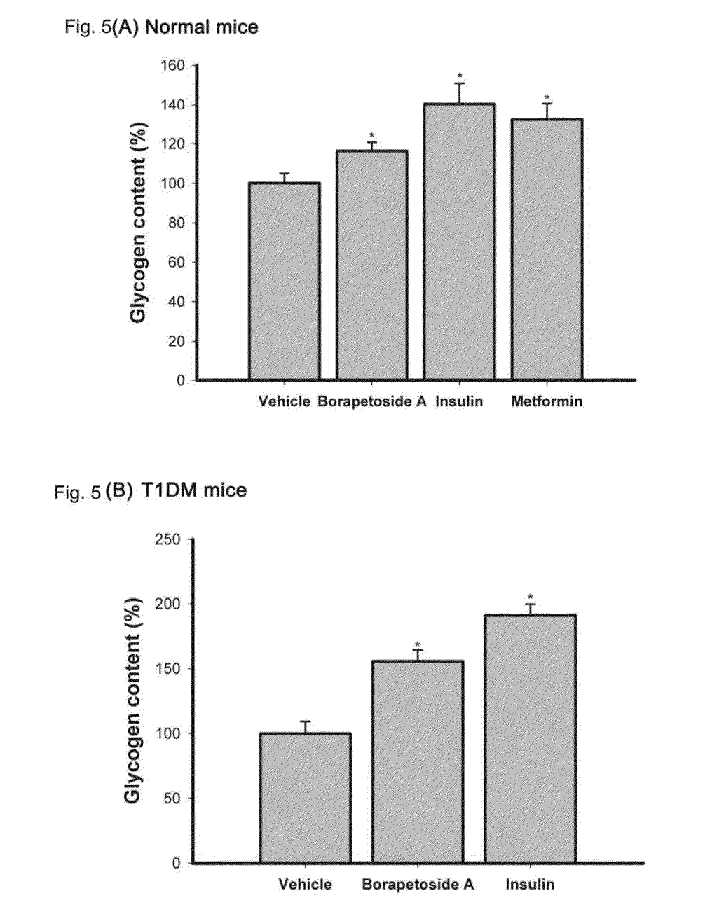

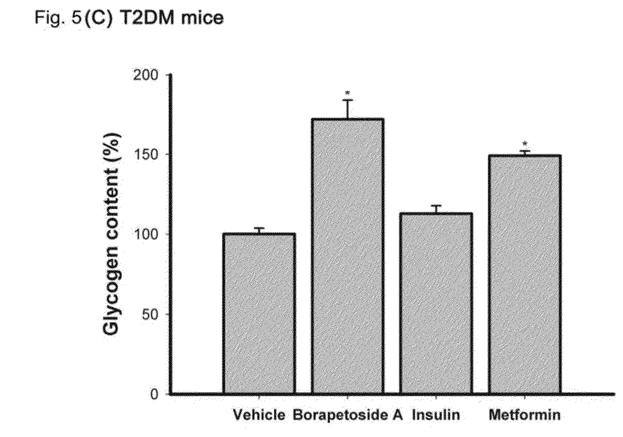

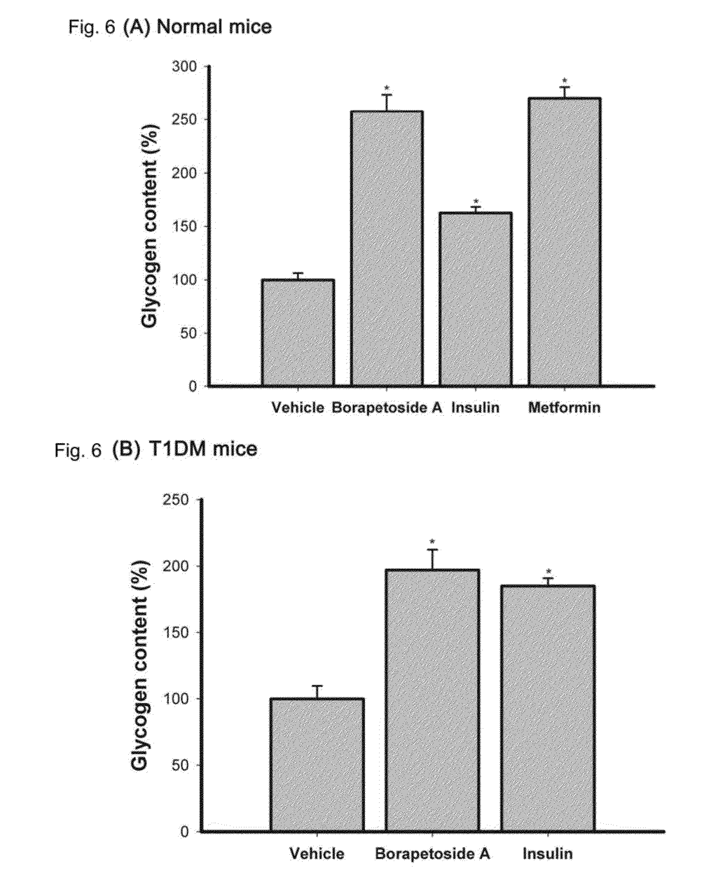

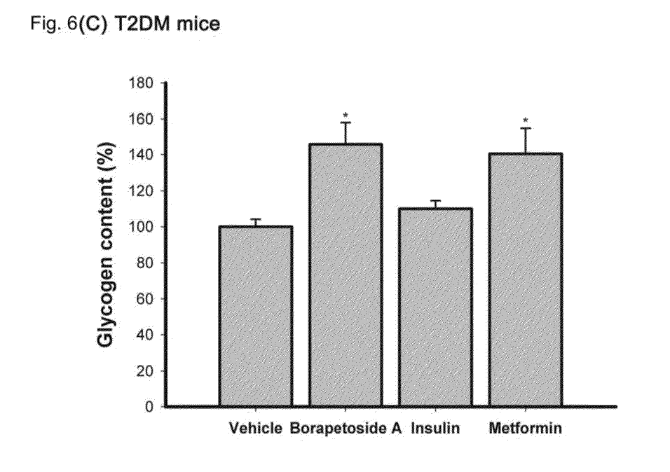

Glucose transport is the rate-limiting step in carbohydrate metabolism. A family of glucose transporters (GLUT) mediates the glucose transport across the cell membrane. Both the GLUT4 in skeletal muscle and GLUT2 in liver are the insulin sensitive glucose transporters. Relative to short-acting insulin, borapetodie A increased similar glycogen synthesis in soleus muscle in the mice with or without DM (FIG. 5). In addition to the skeletal muscle, liver is another important metabolic organ for glucose metabolism. Hepatic insulin resistance is well recognized as the primary leading cause for DM development. In this study, borapetoside A also increased the glycogen synthesis in liver in the mice with or without DM (FIG. 6). Insulin, however, failed to enhance the glycogen synthesis in liver and soleus muscle of the mice with type 2 DM (FIGS. 5C and 6C).

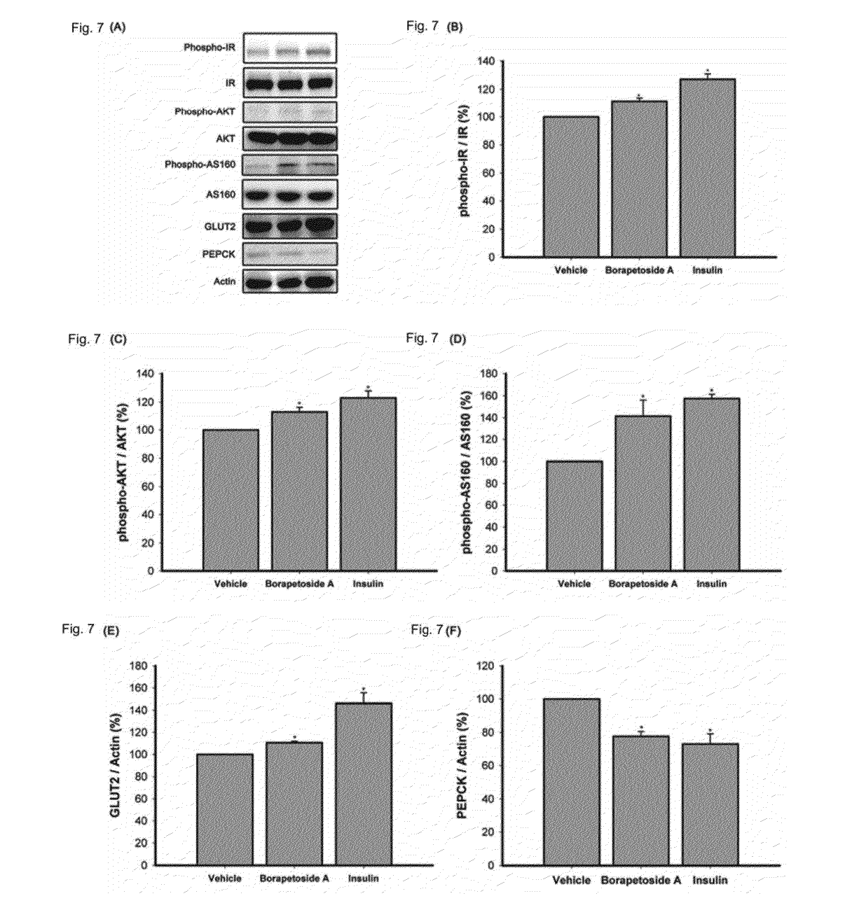

The pathways by which insulin regulates the glucose uptake and carbohydrate metabolism in muscle, fat, and liver tissues, are now well established. The canonical pathway of insulin signaling is via activation of phosphoinositide 3-kinase (PI3K) and Akt, and accessory pathways that contributed to specific insulin action. Insulin initiated the signaling transduction through IR, a large transmembrane protein. Then Akt plays a crucial role in the hepatic insulin signal transduction, in terms of glycogen synthesis. The current evidence suggests that phosphorylation of AS160 is a key regulators of insulin enhanced glucose uptake. Moreover, Akt substrate of 160 kDa, AS160, has strong implication in the regulation of glucose transportation in muscle, adipose tissue, and liver. As shown in FIG. 6, it was found that a seven-day treatment with borapetoside A increased the phosphorylation of IR, Akt, and AS160 as well as the protein expression of GLUT2 in liver in the mice with type 1 DM. Furthermore, the expression of PEPCK was significantly decreased in liver. PEPCK, is one of the key enzymes of hepatic carbohydrate metabolism and catalyzes a regulatory step in gluconeogenesis. The insulin deficiency is clearly associated with the changes in hepatic metabolism, including increased expression of PEPCK. The insulin levels of the mice with type 1 DM is limited, meanwhile the PEPCK gene is overexpressed in liver. Seven-day treatment of borapetoside A in the mice with type 1 DM decreased the expression of PEPCK protein in liver (FIG. 7).

Without binding to any particular theory of mechanism, these findings suggest that borapetoside A exerts an excellent glucose-lowering effect through an enhancement of glucose utilization of skeletal muscle and liver. The therapeutic efficacy of borapetoside A is mediated via the stimulation of IR/AKT/AK160/GLUT2 pathway, and the suppression of PEPCK expression which then contribute to the reduction of the hepatic gluconeogenesis.

In some embodiments provide methods for treating insulin resistant diabetes in a subject comprising administering an effective amount of borapetoside A, or a pharmaceutically acceptable salt, metabolite, solvate or prodrug thereof, to said subject. In some embodiments provide methods of treating diabetes in a subject wherein said subject is in an inflammatory condition comprising administering said subject an effective amount of borapetoside A, or a pharmaceutically acceptable salt, metabolite, solvate or prodrug thereof, to said subject. In some embodiment, the inflammatory condition is induced by IL-6. In certain embodiments, borapetoside A induces increase of glycogen. In certain embodiments, borapetoside A lowers plasma glucose concentration in said subject. In some embodiments, borapetoside A increases the phosphorylation of IR, Akt, and AS160 in liver of said subject (e.g., a subject with type 1 diabetes). In certain embodiments, borapetoside A decreases the expression of PEPCK protein in liver of the subject (e.g., a subject with type 1 diabetes). In certain embodiments, borapetoside A increases the insulin secretion of said subject (e.g., a subject with type 2 diabetes).

In some embodiments provided herein are methods for the treatment of a patient whose diabetes is insulin resistant or in an inflammatory condition comprising administering the patient in need thereof with borapetoside A, or a pharmaceutically acceptable salt, metabolite, solvate or prodrug thereof.

In other embodiments, borapetoside A is isolated from the extracts of Tinospora crispa.

### CERTAIN PHARMACEUTICAL AND MEDICAL TERMINOLOGY

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of“or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

) The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound (i.e., borapetoside A described herein) and a co-agent, are both administered to a patient simultaneously in the form of a single entity or dosage. The term “non-fixed combination” means that the active ingredients, e.g. a compound (i.e., borapetoside A described herein) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound (i.e., borapetoside A described herein) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Routes of Administration and Dosage

Suitable routes of administration include, but are not limited to, oral, intravenous, rectal, aerosol, parenteral, ophthalmic, pulmonary, transmucosal, transdermal, vaginal, optic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, borapetoside A as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In some embodiments, borapetoside A, or a pharmaceutically acceptable salt, metabolite, solvate or prodrug thereof, is administered parenterally or intravenously. In other embodiments, borapetoside A, or a pharmaceutically acceptable salt, metabolite, solvate or prodrug thereof, is administered by injection. In some embodiments, borapetoside A, or a pharmaceutically acceptable salt, metabolite, solvate or prodrug thereof, is administered orally.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the compounds may be given continuously or temporarily suspended for a certain length of time (i.e., a “drug holiday”).

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human.

The dosage of such compounds lies preferably within a range of circulating concentrations that include the ED₅₀ with minimal toxicity. The dosage may vary within this range depending upon the dosage form employed and the route of administration utilized.

### Pharmaceutical Formulation

In some embodiments provide pharmaceutical compositions comprising a therapeutically effective amount of borapetoside A; or a pharmaceutically acceptable salt, metabolite, solvate or prodrug thereof; and a pharmaceutically acceptable excipient.

In some embodiments, borapetoside A described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound (i.e., borapetoside A) and a pharmaceutically acceptable diluent(s), excipient(s), or carrier(s). In certain embodiments, the compounds described are administered as pharmaceutical compositions in which a compound (i.e., borapetoside A described herein) is mixed with other active ingredients, as in combination therapy. Encompassed herein are all combinations of actives set forth in the combination therapies section below and throughout this disclosure. In specific embodiments, the pharmaceutical compositions include one or more compounds (i.e., borapetoside A described herein).

A pharmaceutical composition, as used herein, refers to a mixture of a compound (i.e. borapetoside A described herein) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds (i.e., borapetoside A described herein) are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, a compound (i.e., borapetoside A described herein) is formulated in an aqueous solution. In specific embodiments, the aqueous solution is selected from, by way of example only, a physiologically compatible buffer, such as Hank's solution, Ringer's solution, or physiological saline buffer. In other embodiments, a compound (i.e., borapetoside A described herein) is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions. In specific embodiments, such solutions include physiologically compatible buffers and/or excipients.

In another embodiment, compounds described herein are formulated for oral administration. Compounds described herein, including a compound (i.e., borapetoside A described herein), are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions of a compound (i.e., borapetoside A described herein) are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In one aspect, borapetoside A is prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes a compound (i.e., borapetoside A described herein) to be injected. In general, automatic injectors include a reservoir for holding the solution, which is in fluid communication with a needle for delivering the drug, as well as a mechanism for automatically deploying the needle, inserting the needle into the patient and delivering the dose into the patient. Exemplary injectors provide about 0.3 mL, 0.6 mL, 1.0 mL or other suitable volume of solution at about a concentration of 0.5 mg to 50 mg of a compound (i.e. borapetoside A described herein) per 1 mL of solution. Each injector is capable of delivering only one dose of the compound.

In still other embodiments, borapetoside A is administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, borapetoside A is formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of borapetoside A is accomplished by means of iontophoretic patches and the like. In certain embodiments, transdermal patches provide controlled delivery of borapetoside A. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of a compound (i.e., borapetoside A described herein); (2) a penetration enhancer; and (3) an aqueous adjuvant. In addition, transdermal formulations can include additional components such as, but not limited to, gelling agents, creams and ointment bases, and the like. In some embodiments, the transdermal formulations further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the compounds (i.e., borapetoside A described herein) are formulated for administration by inhalation. Various forms suitable for administration by inhalation include, but are not limited to, aerosols, mists or powders. Pharmaceutical compositions of a compound (i.e., borapetoside A described herein) are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatins for use in an inhaler or insufflator are formulated containing a powder mix of the compound and a suitable powder base such as lactose or starch.

Intranasal formulations are known in the art and are described in, for example, U.S. Pat. Nos. 4,476,116, 5,116,817 and 6,391,452, each of which is specifically incorporated herein by reference. Formulations, which include a compound (i.e. borapetoside A described herein), which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein, may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

In still other embodiments, borapetoside A is formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients is optionally used as suitable and as understood in the art. Pharmaceutical compositions comprising a compound (i.e., borapetoside A described herein) may be manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound (i.e., borapetoside A described herein) described herein as an active ingredient. The active ingredient is in free-acid or free-base form, or in a pharmaceutically acceptable salt form. In addition, the methods and pharmaceutical compositions described herein include the use crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least compound (i.e., borapetoside A described herein) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers. e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of a compound (i.e., borapetoside A described herein). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v, polysorbate 20, (h) arginine, (i) heparin, (j) dextran sulfate, (k) cyclodextrins, (1) pentosan polysulfate and other heparinoids, (m) divalent cations such as magnesium and zinc: or (n) combinations thereof.

It is understood that in some embodiments, the dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in other embodiments, the dosage regimen actually employed varies widely and therefore deviates from the dosage regimens set forth herein.

### EXAMPLES

### Example 1

### Plant Material and Preparation of Plant Extracts

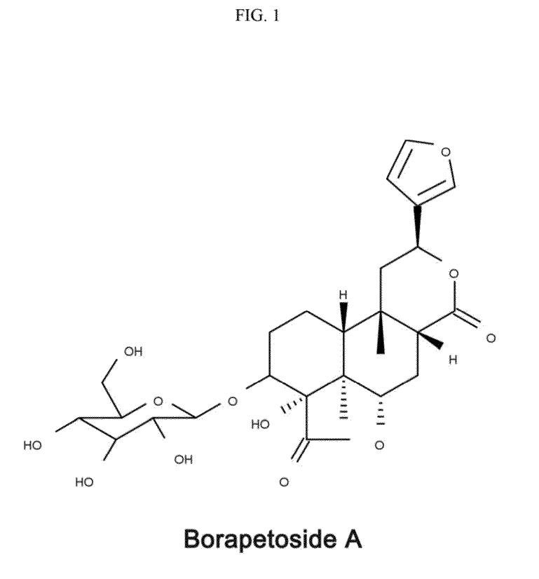

The vines of Tinospora crispa Miers (Menispermaceae) were collected and grounded. Borapetoside A (see FIG. 1) was extracted by the known method (Lam et al. 2012). Alternative methods may be used to prepare Borapetoside A.

### Example 2

### Cell Lines and Cell Culture Preparation

The C2C12 skeletal muscle cells were cultured in Dulbecco's modified Eagle's medium (DMEM; Gibco/Invitrogen, Carlsbad, Calif.), and the human hepatocellular carcinoma cell line, Hl-ep3B, was cultured in Roswell Park Memorial Institute medium 1640 (RPMI 1640 medium; Gibco/Invitrogen, Carlsbad, Calif.). Cells were cultured at 37° C. in the humidified incubator supplied with 5% CO₂. Culture mediums were supplemented with 4.5 mgmL⁻¹ glucose, penicillin 100 IUmL⁻¹, streptomycin 100 μgmL⁻¹ and 10% fetal bovine serum (Gibco/Invitrogen, Carlsbad, Calif.). Only C2C12 cells were then switched to 2% horse serum for 3 days after reaching the 70% confluence. Myotubes formation was achieved after 4 days of incubation, and the cells were used for subsequent experiments. Culture mediums were replaced by serum free DMEM or RPMI for 24 h before experiments (see e.g., procedure described in Sultan et al., 2006).

### Example 3

### Glycogen Content Assay in Cultured Cells

C2C12 and Hep3B cells were treated with 1 nM insulin, 100 μM metformin, and the assigned concentration of borapetoside A for 30 minutes. To develop the insulin resistance state of cells, C2C12 cells were incubated with IL-6 at 20 ngmL⁻¹ for 1 hour. Later, the cells were treated with 1 nM insulin, 100 μM metformin, and the assigned concentration of borapetoside A for 30 minutes. After the assigned treatment, cells were washed twice with phosphate buffered saline (PBS). Glycogen content was measured as previously described, with some modifications (see e.g., Savage et al., 2008). The protein concentrations of the cell lysates were determined by using Pierce BCA Protein Assay Kit (Thermo-Scientific, Rockford, Ill.). The glycogen content of each sample was normalized by total protein.

### Example 4

### Animal Study of Borapetoside a Treatment Re Plasma Glucose and Insulin Level

The 4-week-old male ICR mice were purchased from the Animal Center of the College of Medicine, National Taiwan University (Taipei, Taiwan) with delicate humane care. This animal study was conducted by following the University ethical guidelines and the ‘Guide for the Care and Use of Laboratory Animals’ published by the US National Institutes of Health (NIH publication no. 85-23, revised 1996). The animal experiments were approved by the Institutional Animal Care and Use Committee (IACUC) of the National Taiwan University (IACUC No. 20110073). Animals were housed in a room at the constant temperature, 22±1° C., with 12 hours light and dark cycles.

After three days of acclimatization, the mice had free access to a standard rodent chow (normal mice), a standard rodent chow after single 250 mgkg⁻¹ streptozotocin (STZ) intraperitoneal injection (TIDM mice), and a fat-rich chow diet and fructose-sweetened water (T2DM mice) for 4 weeks. The induction of Type 1 DM was assessed and confirmed when the mice had plasma glucose levels ≧350 mgdL⁻¹, accompanied with polyuria, hyperphagia and decreased body weight. The induction Type 2 DM in the mice was assessed by measuring fasting plasma glucose levels and confirmed when plasma glucose level was ≧150 mgdL⁻¹ after the 4-week induction. The animals were assigned randomly into three groups, with seven animals in each group. In group I, the mice were treated with vehicle, 2% DMSO in normal saline, as the negative control. In group II, the mice were intraperitoneally treated with borapetoside A (0.1-10 mgkg⁻¹). In group II, the positive control, the mice received intraperitoneally metformin (300 mgkg⁻¹; Sigma Chemical Co., St. Louis, Mo., U.S.A.) or insulin (0.5 IUkg⁻¹; Insulin Actrapid® HM; Novo Nordisk, Denmark). In the acute study, mice under anesthesia received a single intraperitoneal administration of borapetoside A, negative control, or positive control. In the subacute study, mice received intraperitoneal administrations of borapetoside A, vehicle, or insulin twice a day for seven days.

Blood samples were collected from the orbital vascular plexus of the mice under anesthesia. Blood samples were then centrifuged at 13000 rpm for 5 min, and the plasma samples were collected and kept on ice prior to the assay. The plasma glucose concentration was measured by Glucose Kit Reagent (Biosystems S.A., Barcelona, Spain) following the manufacturer's instructions, and the insulin levels were estimated using mouse insulin ELISA kit (Mercodia AB, Uppsala, Sweden).

The mice were maintained under anesthesia throughout the procedure. Glucose (2.0 gkg⁻¹) was administered intraperitoneally. Blood samples were collected from the orbital vascular plexus at 0, 30, 60, 120 min. The concentrations of the plasma glucose were measured, and the values of AUC were calculated accordingly.

The mice were injected with borapetoside A (10 mgkg⁻¹, interperitoneal) for 60 minutes or insulin (0.5 IUkg⁻¹, interperitoneal) for 30 minutes. After the treatment, the anesthetic mice were sacrificed by cervical dislocation to collect the soleus muscle and liver. About 40 mg of each sample was dissolved in 1 mol/L KOH at 75° C. for 30 min. The dissolved homogenate was neutralized by glacial acetic acid and then incubated overnight in acetate buffer (0.3 mol/L sodium acetate, pH 4.8) containing amyloglucosidase (Sigma, St. Louis, Mo.). The mixture was then neutralized with 1 mol/L NaOH to stop the reaction. The glycogen contents in the tissue samples were determined as μg of glucose per mg of tissue (wet weight).

### Western Blot Assay and Analysis

Western blot analysis was carried out by known methods with a slight modification. Briefly, tissues were homogenized in T-PER® Tissue Protein Extraction Reagent (Pierce Biotechnology, Rockford, Ill.) with Halt™ Protease Inhibitor Single-Use Cocktail (Pierce Biotechnology, Rockford, Ill.). Liver homogenates were prepared by mechanical homogenization (Polytron PT3100, Luzernerstrasse, Switzerland). After centrifuging the homogenates at 10,000 g for 30 minutes, the protein concentrations were determined by using a BCA Protein Assay Kit (Pierce Biotechnology, Rockford, Ill.). Later, 60 μg of protein preparations were applied on 10% sodium dodecyl sulfate-polyacrylamide gel for electrophoresis and then transferred to polyvinylidene difluoride membranes (Millipore, Billerica, Mass.). The membranes were analysed by using the antibodies against phospho-insulin receptor β (IRβ) (Tyr¹³⁴⁵), phospho-Akt (Ser⁴⁷³), phosphor-AS160 (Thr⁶⁴²), AS160 (Cell Signaling Technology, Beverly, Mass.), IRβ, Akt, β-Actin (Santa Cruz Biotechnology, Santa Cruz, Calif.), GLUT2 (Abcam, Cambridge, UK), and PEPCK (a gift from Professor D K Granner), respectively. Chemiluminescence detection and image analysis was performed with UVP-Biochimie Bioimager and LabWorks software (UVP, Upland, Calif.). The intensity of each band was quantified with ImageQuant.

### Statistical Analysis of the Data

The results are presented as mean±the standard error of the mean (SEM). Statistical difference between the means of the various groups were analyzed using one way analysis of variance (ANOVA), followed by Turkey's multiple test with Prism 5.0 demo software (GraphPad Software Inc., CA). Results were considered statistical significant at the 95% confidence interval (i.e., p<0.05).

### Results and Analysis

To investigate the influence of borapetoside A treatment on the uptake of glucose in metabolic organs and tissues, such as the liver and skeletal muscles, the C2C 12 myotube and Hep3B hepatocytes were exposed to borapetoside A at the assigned concentrations. As shown in FIG. 2A, when differentiated C2C12 myotubes were stimulated with 10⁻¹, 10⁻⁷, and 10⁻⁶ mol/L borapetoside A, there were 1.53±0.10-, 1.28±0.04-, 1.49±0.15-fold increase of glycogen, respectively. In the IL-6-induced insulin resistant model, glycogen content was not increased by insulin treatment. However, the glycogen contents of the IL-6-treated cells were increased by 1.35±0.08-, 1.38±0.06-, 1.64±0.05-fold while receiving the 10⁻⁸, 10⁻⁷, and 10⁻⁶ mol/L borapetoside A treatment, respectively (FIG. 2B). Hepatocyte Hep3B stimulated with 10⁻⁷, 10⁻⁶ and 10⁻⁵ mol/L borapetoside A showed a 1.46±0.05-, 1.39±0.12-, 1.54±0.11-fold increase of glycogen content (FIG. 2C).

Thus it is clearly shown that Borapetoside A induced glycogen accumulation in C212 and Hep3B and thus enhances glycogen synthesis.

The effects of borapetoside A on blood glucose and insulin levels were measured and analyzed. In FIG. 3, the antihyperglycemic effects of borapetoside A and the reference drug, metformin, was analyzed at 60 min after a single dose administration in the normal, type 1 DM, and type 2 DM animal models, respectively. Borapetoside A was administered to the normal mice, the mice with type 1 DM, and the mice with type 2 DM at dosages of 0.1, 0.3, 1.0, 3.0, and 10.0 mgkg⁻¹. The plasma glucose level of the treated mice was examined at 0 and 60 minutes after the treatment. Borapetoside A lowered the plasma glucose level in normal mice, the type 1 DM and type 2 DM bearing mice in a dose-dependent manner. A significant decrease of plasma glucose level and the mice with type 2 DM were observed when the dose of borapetoside A was higher than 0.3 mgkg⁻¹. In the normal mice, the effect was not observed until the treatment with 1 mgkg⁻¹ of borapetoside A. As such, it is clearly shown that Borapetoside A could stimulate insulin release in the normal mice and the mice with type 2 DM but not in those with type 1 DM (Table 1). The plasma insulin level was dose-dependently increased at the dose range between 0.3 and 10 mgkg⁻¹ by borapetoside A in the normal mice. In contrast, in the mice with type 2 DM group, the concentration of plasma insulin was increased only by 3.0 and 10 mgkg⁻¹ of borapetoside A, However, in the mice with type 1 DM, the insulin-secretion deficient model animals, neither the borapetoside A nor metformin could rescue the plasma insulin level.

TABLE 1
The plasma insulin levels in normal and
diabetic mice before and after treatment

| | Plasma insulin (pmol/l) | |
|---|---|---|
| Groups | Before-treatment | After-treatment |
| Normal mice vehicle borapetoside A | 66.8 ± 2.4 | 67.8 ± 3.0 |
| 0.1 mg kg⁻¹ | 61.0 ± 4.6 | 68.3 ± 4.4 |
| 0.3 mg kg⁻¹ | 63.4 ± 3.9 | 129.4 ± 22.0* |
| 1 mg kg⁻¹ | 66.1 ± 5.4 | 118.8 ± 10.1* |
| 3 mg kg⁻¹ | 64.4 ± 3.6 | 121.2 ± 14.2* |
| 10 mg kg⁻¹ | 68.9 ± 1.3 | 108.2 ± 2.9* |
| Metformin | 66.6 ± 1.3 | 72 ± 3.1 |
| T1DM mice vehicle borapetoside A | 22.8 ± 0.2 | 22.8 ± 0.1 |
| 0.1 mg kg⁻¹ | 22.9 ± 0.1 | 22.7 ± 0.2 |
| 0.3 mg kg⁻¹ | 22.9 ± 0.1 | 22.7 ± 0.3 |
| 1 mg kg⁻¹ | 23.1 ± 0.2 | 23.0 ± 0.2 |
| 3 mg kg⁻¹ | 23.1 ± 0.2 | 23.0 ± 0.0 |
| 10 mg kg⁻¹ | 23.1 ± 0.1 | 23.0 ± 0.1 |
| Metformin | 22.4 ± 0.4 | 22.7 ± 0.3 |
| T2DM mice vehicle borapetoside A | 139.1 ± 2.1 | 141.7 ± 2.3 |
| 0.1 mg kg⁻¹ | 140.5 ± 4.1 | 134.5 ± 9.3 |
| 0.3 mg kg⁻¹ | 133.0 ± 4.2 | 149.0 ± 9.5 |
| 1 mg kg⁻¹ | 126.8 ± 5.7 | 123.7 ± 9.6 |
| 3 mg kg⁻¹ | 138.3 ± 3.6 | 184.3 ± 14.6* |
| 10 mg kg⁻¹ | 134.9 ± 3.5 | 205.3 ± 22.2* |
| Metformin | 148.1 ± 15.2 | 182.0 ± 27.7 |

Values were expressed as mean ± SEM from six animals in each group.
*p < 0.05 from vehicle group vs drug-treated.

The influence of borapetoside A on glucose tolerance test were measured and analyzed. The influence of borapetoside A on plasma glucose levels in the normal mice and the mice with type 2 DM was first investigated. As shown in FIG. 4A, the concentrations of plasma glucose increased at 30 min after the administration of dextrose in all groups and decreased thereafter in both the normal mice and the mice with type 2 DM. The increase of plasma glucose level at 30 min was significantly suppressed in borapetoside A-treated (0.1, 1, and 10 mgkg⁻¹) and metformin-treated groups, compared to the vehicle group. The area under the curve (AUC) of borapetoside A (0.1, 1, and 10 mgkg⁻¹) also dose-dependently decreased to 78.8±3.0%, 69.9±7.1%, and 68.91±3.5% of vehicle control in normal mice, and 74.9±4.5, 71.1±3.2, and 68.9±1.9% of vehicle control in type 2 DM bearing mice respectively (FIGS. 4B and D). These data indicated that glucose tolerance was improved in the borapetoside A-treated group.

The influence of borapetoside A on glycogen synthesis in the isolated soleus muscle and the liver was measured and analyzed. To further characterize the effects of borapetoside A treatment on glycogen metabolism, glycogen content of each treated groups was assessed. All three groups, the normal mice, the mice with type 1 DM, and the mice with type 2 DM, were intraeritoneally injected with borapetoside A, Actrapid or metformin. Their glycogen contents in liver and soleus muscle were determined 60 minutes after the injections. As shown in FIG. 5A-C, in soleus muscle, the glycogen content was significantly increased to 116.5±4.2%, 155.6±8.6%, and 171.8±12.0%, of vehicle control in the borapetoside A treated normal mice, mice with type 1 DM, and mice with type 2 DM, respectively. Furthermore, regarding to major metabolic organ, liver, borapetoside A treatment significantly resulted in an increase of glycogen content to 257.8±15.8%, 196.9±15.6%, and 146.1±12.0% of vehicle control in the normal mice, the mice with type 1 DM, and the mice with type 2 DM (FIG. 6A-C).

The influence of borapetoside A on the insulin signaling pathway in the livers of the mice with type 1 DM was assessed. To characterize the molecular effects of borapetoside A on glucose utilization in liver, the phosphorylation status of IR, Akt/PKB, and AS160 and the expression profile of GLUT2 and PEPCK in liver tissues was further examined via western blot analysis. The liver tissues were collected from the mice with type 1 DM receiving borapetoside A treatment at a dosage of 10 mgkg⁻¹ or receiving insulin treatment twice a day for 7 days. The results showed that borapetoside A treatment increased the phosphorylated IR, Akt, and AS160, and it also increased the expression of GLUT2 in the liver tissues of the mice with type 1 DM (FIG. 7A). Moreover, the significant decrease of PEPCK expression was observed in the liver tissue. The density ratios of phosphor-Tyr¹³⁴⁵-IR to IR, phosphor-Ser⁴⁷³-Akt to Akt, phosphor-Thr⁶⁴²-AS160 to AS160, GLUT2 to β-actin and PEPCK to β-actin were calculated and plotted in the FIG. 7B-7F.

### Example 5

### Oral Formulation

To prepare a pharmaceutical composition for oral delivery, 100 mg of an exemplary borapetoside A was mixed with 100 mg of corn oil. The mixture was incorporated into an oral dosage unit in a capsule, which is suitable for oral administration.

In some instances, 100 mg of borapetoside A is mixed with 750 mg of starch. The mixture is incorporated into an oral dosage unit for, such as a hard gelatin capsule, which is suitable for oral administration.

### Example 6

### Sublingual (Hard Lozenge) Formulation

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 7

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating insulin resistant diabetes in a subject comprising administering an effective amount of borapetoside A, or a pharmaceutically acceptable salt, metabolite, solvate or prodrug thereof, to said subject.

2. The method of claim 1, wherein said insulin resistant diabetes is type 2 diabetes.

3. The method of claim 1, wherein borapetoside A induces increase of glycogen.

4. The method of claim 1, wherein borapetoside A lowers plasma glucose concentration in said subject.

5. The method of claim 1, wherein borapetoside A increases the phosphorylation of IR, Akt, and AS160 in liver of said subject.

6. The method of claim 1, wherein borapetoside A decreases the expression of PEPCK protein in liver of the subject.

7. A method of claim 1, wherein borapetoside A increases insulin secretion of said subject.

8. A method for treating diabetes in a subject wherein said subject is in an inflammatory condition comprising administering said subject an effective amount of borapetoside A, or a pharmaceutically acceptable salt, metabolite, solvate or prodrug thereof, to said subject.

9. The method of claim 8, wherein said inflammatory condition is induced by IL-6.

10. The method of claim 8, wherein borapetoside A induces increase of glycogen.

11. The method of claim 8, wherein borapetoside A lowers plasma glucose concentration in said subject.

12. The method of claim 8, wherein borapetoside A increases the phosphorylation of IR, Akt, and AS160 in liver of said subject.

13. The method of claim 12, wherein borapetoside A decreases the expression of PEPCK protein in liver of the subject with type 1 diabetes.

14. A method of claim 8, borapetoside A increases the insulin secretion of said subject.
